(19) United States
(12) Patent Application Publication
(10) Pub. No.: US 2008/0234532 A1
(43) Pub. Date: Sep. 25, 2008

(54) FIDUCIAL MARKER

(75) Inventors: Mark De Langen, Rotterdam (NL); Stuart Green, Grimsargh (GB); Jorge Schlegel, Albstadt (DE)

Correspondence Address: MICHAEL BEST & FRIEDRICH LLP, 100 E WISCONSIN AVENUE, Suite 3300, MILWAUKEE, WI, 53202, US

(73) Assignee: INVIBIO LIMITED, Lancashire (GB)

(21) Appl. No.: 12/105,498

(22) Filed: Apr. 18, 2008

Related U.S. Application Data
(63) Continuation-in-part of application No. PCT/GB2006/003947, filed on Oct. 23, 2006.

(30) Foreign Application Priority Data
Oct. 22, 2005 (GB) 0521536.3
Apr. 20, 2007 (GB) 0707671.4

(51) Int. Cl. A61M 36/12

(52) U.S. Cl. 600/8

(57) ABSTRACT

A fiducial marker which is visible to a wide range of imaging techniques, comprises a radiopaque material, such as barium sulphate or a metal wire, encapsulated in a biocompatible polymeric material, for example a polyaryletherketone such as polyetheretherketone.

### RELATED APPLICATIONS

This application is a continuation-in-part of International Application No. PCT/GB2006/003947, filed Oct. 23, 2006. This application also claims the benefit of Great Britain Patent Application No. 0707671.4, filed Apr. 20, 2007.

This patent application fully incorporates by reference the subject matter of each of the above-identified patent applications to which this application claims priority. The entire disclosure of each patent application is considered to be part of the accompanying application.

### BACKGROUND OF THE INVENTION

This invention relates to fiducial markers.

Visualisation techniques such as computer tomographic (CT) X-ray imaging and magnetic resonance imaging (MRI) machines are now well-known systems for imaging structures of the human body for subsequent assessment by a clinician to establish if any abnormalities are present. In the event of any abnormalities, for example a cancer, being noted the body may be subjected to focused treatment to remove or destroy the abnormality, for example using chemotherapy, radiation therapy and/or surgery.

In chemotherapy, drugs are used to destroy the abnormality. During the course of a treatment visualisation techniques are used to monitor the progress of the treatment and the effect of the treatment can be assessed by comparison of images taken over the course of the treatment.

In radiation therapy, images of the abnormality are used by a radiologist to adjust the irradiating device and to direct radiation solely at the abnormality while minimizing or eliminating adverse effects to surrounding healthy tissue. During the course of the radiation treatment, visualization techniques are used to follow the progress of the treatment.

When surgery is used to remove an abnormality, the images of the lesion in the patient can guide the surgeon during the operation. By reviewing the images prior to surgery, the surgeon can decide the best strategy for reaching and biopsying, excising, or otherwise manipulating the abnormality. After surgery has been performed, further scanning is utilized to evaluate the success of the surgery and the subsequent progress of the patient.

It will be appreciated from the above that there is a need associated with the aforementioned visualization techniques and/or treatments to provide a means of accurate selection and comparison of views of identical areas in images which have been obtained by imaging techniques at different times or at the same time using two or more different imaging techniques, such as both CT and MRI techniques. It is known to use fiducial markers to address the aforementioned problems. Such markers are artificial markers which are introduced into a human body and fixed in position by a surgeon at or adjacent an abnormality to provide a clear and accurate reference point which is visible on scans produced using visualization techniques such as CT and MRI techniques.

It is known to use markers in the form of wire or beads made of highly radiopaque materials such as gold or tantalum. However, there are problems associated with such materials. For example it is found that in a CT scan, the gold or tantalum marker may lead to production of artefacts in the image produced, for example information may be missing and/or “starbursts” may be present, leading to difficulties in accurately interpreting the images. Also, in MRI techniques, eddy currents may be produced in the gold or tantalum which again may result in the production of artefacts which render image interpretation more difficult.

### SUMMARY OF THE INVENTION

It is desirable that any fiducial marker is visible under MRI, CT and X-ray imaging so that, in any situation, one or more of the techniques may be used to visualize any marker.

It is also desirable to use fiducial markers which are as small as possible, to minimise patients' discomfort. On the other hand clinicians require markers to provide a strong signal which implies such markers should be as large as possible.

It is an object of the present invention to address problems associated with fiducial markers.

It is an object of the present invention to provide a fiducial marker which is small enough to be left in a patients' body with minimum discomfort and yet which is clearly visible under a range of imaging techniques, such as CT, MRI and conventional X-ray techniques with minimal artefacts such as starbursts.

According to a first aspect of the invention, there is provided a fiducial marker which comprises a radiopaque material encapsulated in a bio-compatible polymeric material.

Said marker suitably has a maximum dimension measured in a first direction of less than 50 mm. In this case, a marker may be elongate, for example in the form of a string or the like. Suitably, said marker has a maximum dimension measured in a first direction of less than 10 mm, preferably less than 8 mm, more preferably less than 6 mm, especially less than 4 mm. The maximum dimension may be at least 1 mm or at least 2 mm. Typically, the maximum dimension in said first direction may be in the range 1.5 to 4 mm.

Said marker preferably has a dimension in a second direction perpendicular to the first direction which is less than said maximum dimension in said first direction. The ratio of the maximum dimension in said first direction to said dimension in said second direction may be greater than 1, preferably greater than 1.1, more preferably greater than 1.3, especially greater than 1.5. The ratio may be less than 5, preferably less than 4, more preferably less than 3, especially less than 2.

The volume of the marker may be less than 20 mm³, suitably less than 15 mm³, preferably less than 10 mm³, more preferably less than 8 mm³, especially less than 6 mm³. The volume may be at least 0.75 mm³, preferably at least 1 mm³.

The density of the marker may be at least 1.1 g/cm³, suitably at least 1.2 g/cm³, preferably at least 1.3 g/cm³, more preferably at least 1.5 g/cm³, especially at lest 1.6 g/cm³. The density may be less than 3.5 g/cm³, suitably less than 3.2 g/cm³. Typically the density may be in the range 1.5 to 3 g/cm³.

Said marker preferably has a substantially constant cross-section along at least 50%, suitably at least 70%, preferably at least 90%, more preferably at least 95%, especially about 100%, of its extent in one direction, for example said first direction referred to. Said cross-section is preferably substantially symmetrical about a first plane which bisects the cross-section in one direction; preferably also it is symmetrical about two mutually orthogonal planes which bisect the cross-section. Said cross-section preferably includes a substantially circular outer wall. Said cross-section described may be substantially annular or circular. It preferably includes substantially no void areas.

Said cross-section preferably has an area of less than 5 mm², preferably less than 4mm², more preferably less than 3 mm², especially less than 2 mm². The area may be less than 1.5 mm². The area is preferably greater than 0.5 mm².

Said cross-section is preferably of substantially constant shape on moving from one side of the marker to an opposite side thereof.

In an alternative embodiment, said marker may be substantially spherical.

Said fiducial marker may be in the form of an extruded tube, coil or solid member. Said marker preferably includes substantially no void areas; it is preferably substantially solid throughout.

Said radiopaque material is preferably an integral part of said marker. Said radiopaque material is preferably not flowable within the marker. Said radiopaque material is preferably substantially immovably fixed in position in said marker so that its position relative to that of the polymeric material is substantially immovably fixed.

Said radiopaque material is preferably covered, at least in part, by said bio-compatible polymeric material. Said radiopaque material is preferably substantially fully enclosed by said bio-compatible polymeric material.

Radiopaque material and polymeric material are preferably contiguous. Preferably substantially all of the radiopaque material is contiguous with bio-compatible polymeric material.

Said fiducial marker preferably includes no part which is arranged to be moved, for example pivoted, between predetermined first and second positions. Said marker preferably includes no moving parts. It should be appreciated however that this does not exclude the possibility of the marker being manipulated, for example bent, into any particular shape.

Said fiducial marker preferably comprises radiopaque material and polymeric material which have been extruded.

Said fiducial marker may have a weight of at least 3 mg, preferably at least 5 mg. The weight may be less than 100 mg, suitably less than 75 mg, preferably less than 50 mg, more preferably less than 25 especially less than 10 mg.

Said marker may include at least 1 wt %, suitably at least 3 wt %, preferably at least 10 wt %, more preferably at least 20 wt %, especially at least 30 wt % of radiopaque material. In some embodiments said marker may include at least 35 wt % or at least 40 wt % of said radiopaque material. The amount of radiopaque material may be less than 80 wt %, suitably less than 70 wt %, preferably less than 60 wt %, more preferably 55 wt % or less, especially 50 wt % or less.

Said marker may include at least 30 wt %, preferably at least 40 wt %, more preferably at least 45 wt %, especially at least 50 wt % of said bio-compatible material. The amount of bio-compatible polymeric material may be 97 wt % or less, suitably 90 wt % or less, preferably 80 wt % or less, more preferably 70 wt % or less, especially 65 wt % or less.

The sum of the wt % of said bio-compatible polymeric material and said radiopaque material in said fiducial marker may be at least 60 wt %, suitably at least 70 wt %, preferably at least 80 wt %, more preferably at least 90 wt %, especially at least 99 wt %.

Said bio-compatible polymeric material may be any polymeric material which is non-toxic and not otherwise harmful when introduced into the human or animal body as a fiducial marker.

Said bio-compatible polymeric material may have a Notched Izod Impact Strength (specimen 80 mm×10 mm×4 mm with a cut 0.25 mm notch (Type A), tested at 23° C., in accordance with ISO180) of at least 4 KJm⁻², preferably at least 5 KJm⁻², more preferably at least 6 KJm⁻². Said Notched Izod Impact Strength, measured as aforesaid, may be less than 10 KJm⁻², suitably less than 8 KJm⁻².

The Notched Izod Impact Strength, measured as aforesaid, of the composite material of said fiducial marker may be at least 3 KJm⁻², suitably at least 4 KJm⁻², preferably at least 5 KJm⁻². Said impact strength may be less than 50 KJm⁻², suitably less than 30 KJm⁻².

Said bio-compatible polymeric material suitably has a melt viscosity (MV) of at least 0.06 kNsm⁻², preferably has a MV of at least 0.09 kNsm⁻², more preferably at least 0.12 kNsm⁻², especially at least 0.15 kNsm⁻².

MV is suitably measured using capillary rheometry operating at 400° C. at a shear rate of 1000 s⁻¹ using a tungsten carbide die, 0.5×3.175 mm.

Said bio-compatible polymeric material may have a MV of less than 1.00 kNsm⁻², preferably less than 0.5 kNsm⁻².

Said bio-compatible polymeric material may have a MV in the range 0.09 to 0.5 kNsm⁻², preferably in the range 0.14 to 0.5 kNsm⁻².

Said bio-compatible polymeric material may have a tensile strength, measured in accordance with ISO527 (specimen type 1b) tested at 23° C. at a rate of 50 mm/minute of at least 20 MPa, preferably at least 60 MPa, more preferably at least 80 MPa. The tensile strength is preferably in the range 80-110 MPa, more preferably in the range 80-100 MPa.

Said bio-compatible polymeric material may have a flexural strength, measured in accordance with ISO178 (80 mm×10 mm×4 mm specimen, tested in three-point-bend at 23° C. at a rate of 2 mm/minute) of at least 50 MPa, preferably at least 100 MPa, more preferably at least 145 MPa. The flexural strength is preferably in the range 145-180 MPa, more preferably in the range 145-164 MPa.

Said bio-compatible polymeric material may have a flexural modulus, measured in accordance with ISO178 (80 mm×10 mm×4 mm specimen, tested in three-point-bend at 23° C. at a rate of 2 mm/minute) of at least 1 GPa, suitably at least 2 GPa, preferably at least 3 GPa, more preferably at least 3.5 GPa. The flexural modulus is preferably in the range 3.5-4.5 GPa, more preferably in the range 3.5-4.1 GPa.

Said bio-compatible polymeric material may be amorphous or semi-crystalline. It is preferably semi-crystalline. The level and extent of crystallinity in a polymer is preferably measured by wide angle X-ray diffraction (also referred to as Wide Angle X-ray Scattering or WAXS), for example as described by Blundell and Osborn (Polymer 24, 953, 1983). Alternatively, crystallinity may be assessed by Differential Scanning Calerimetry (DSC).

The level of crystallinity of said bio-compatible polymeric material may be at least 1%, suitably at least 3%, preferably at least 5% and more preferably at least 10%. In especially preferred embodiments, the crystallinity may be greater than 25%.

The main peak of the melting endotherm (Tm) of said bio-compatible polymeric material (if crystalline) may be at least 300° C.

Said bio-compatible polymeric material may include a polymeric moiety which is: an acrylate (e.g. it comprises or consists of methylmethacrylate moieties); a urethane; a vinyl chloride; a silicone; a siloxane (e.g. comprising dimethylsiloxane moieties); a sulphone; a carbonate; a fluoroalkylene (e.g. a fluoroethylene); an acid (e.g. a glycolic acid or lactic acid); an amide (e.g. comprising nylon moieties); an alkylene (e.g. ethylene or propylene); an oxyalkylene (e.g. polyoxymethylene); an ester (e.g. polyethylene terephthalate), an ether (e.g. an aryletherketone, an arylethersulphone (e.g. polyethersulphone or polyphenylenesulphone) or an ether imide).

Said bio-compatible polymeric material may be a resorbable polymer.

Said bio-compatible polymeric material may be selected from a polyalkylacrylate (e.g. polymethylmethacrylate), a polyfluoroalkylene (e.g. PTFE), a polyurethane, a polyalkylene (e.g. polyethylene or polypropylene), a polyoxyakylene (e.g. polyoxymethylene), a polyester (e.g. polyethylene terephthalate or polybutylene terephthalate), a polysulphone, a polycarbonate, a polyacid (e.g. polyglycolic acid or polylactic acid), a polyalkylene oxide ester (e.g. polyethylene oxide terephalate) a polyvinylchloride, a silicone, a polysiloxane, a nylon, a polyaryletherketone, a polarylethersulphone, a polyether imide and any copolymer which includes any of the aforementioned.

Preferably, said bio-compatible polymeric material is selected from resorbable polymers, polyethylene, polypropylene, silicone and polyetheretherketone. More preferably, said polymeric material is selected from polyethylene, polypropylene, silicone and polyetheretherketone.

Said bio-compatible polymeric material may be a homopolymer having a repeat unit of general formula

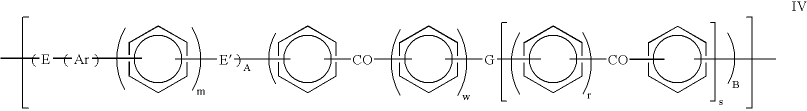

or a homopolymer having a repeat unit of general formula

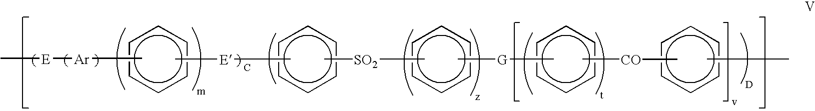

or a random or block copolymer of at least two different units of IV and/or V
wherein A, B, C and D independently represent 0 or 1,

E and E′ independently represent an oxygen or a sulphur atom or a direct link, G represents an oxygen or sulphur atom, a direct link or a —O-Ph-O— moiety where Ph represents a phenyl group, m, r, s, t, v, w, and z represent zero or 1 and Ar is selected from one of the following moieties (i) to (v) which is bonded via one or more of its phenyl moieties to adjacent moieties

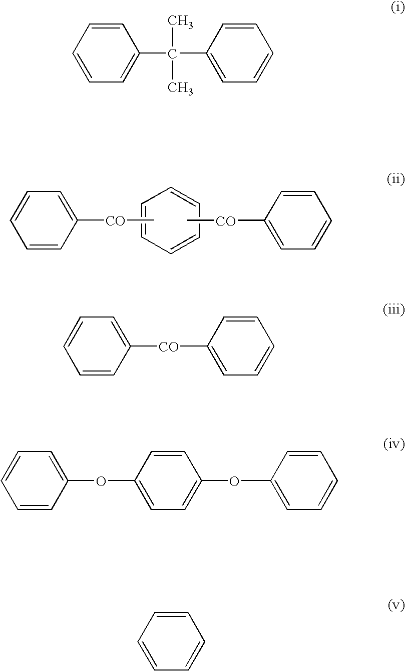

Unless otherwise stated in this specification, a phenyl moiety has 1,4-, linkages to moieties to which it is bonded.

As an alternative to a bio-compatible polymeric material comprising units IV and/or V discussed above, said bio-compatible polymeric material may be a homopolymer having a repeat unit of general formula

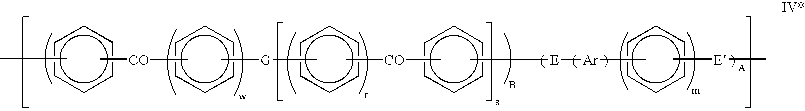

or a homopolymer having a repeat unit of general formula

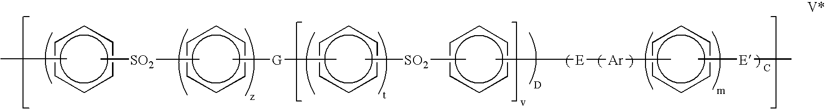

or a random or block copolymer of at least two different units of IV* and/or V*, wherein A, B, C, and D independently represent 0 or 1 and E, E′, G, Ar, m, r, s, t, v, w and z are as described in any statement herein.

Preferably, said bio-compatible polymeric material is a homopolymer having a repeat unit of general formula IV.

Preferably Ar is selected from the following moieties (vi) to (x)

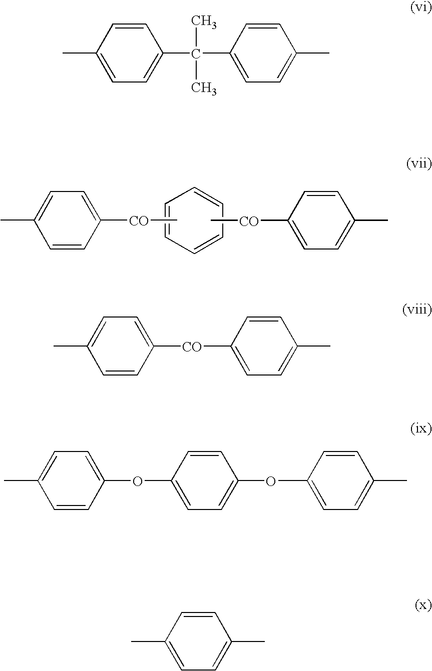

In (vii), the middle phenyl may be 1,4- or 1,3-substituted. It is preferably 1,4-substituted.

Suitable moieties Ar are moieties (ii), (iii), (iv) and (v) and, of these, moieties, (ii), (iii) and (v) are preferred. Other preferred moieties Ar are moieties (vii), (viii), (ix) and (x) and, of these, moieties (vii), (viii) and (x) are especially preferred.

An especially preferred class of bio-compatible polymeric materials are polymers (or copolymers) which consist essentially of phenyl moieties in conjunction with ketone and/or ether moieties. That is, in the preferred class, the first polymer material does not include repeat units which include —S—, —SO₂— or aromatic groups other than phenyl. Preferred bio-compatible polymeric materials of the type described include:
(a) a polymer consisting essentially of units of formula IV wherein Ar represents moiety (v), E and E′ represent oxygen atoms, m represents 0, w represents 1, G represents a direct link, s represents 0, and A and B represent 1 (i.e. polyetheretherketone).
(b) a polymer consisting essentially of units of formula IV wherein E represents an oxygen atom, E′ represents a direct link, Ar represents a moiety of structure (ii), m represents 0, A represents 1, B represents 0 (i.e. polyetherketone);
(c) a polymer consisting essentially of units of formula IV wherein E represents an oxygen atom, Ar represents moiety (ii), m represents 0, E′ represents a direct link, A represents 1, B represents 0, (i.e. polyetherketoneketone).
(d) a polymer consisting essentially of units of formula IV wherein Ar represents moiety (ii), E and E′ represent oxygen atoms, G represents a direct link, m represents 0, w represents 1, r represents 0, s represents 1 and A and B represent 1. (i.e. polyetherketoneetherketoneketone).
(e) a polymer consisting essentially of units of formula IV, wherein Ar represents moiety (v), E and E′ represents oxygen atoms, G represents a direct link, m represents 0, w represents 0, S, r, A and B represent 1 (i.e. polyetheretherketoneketone).
(f) a polymer comprising units of formula IV, wherein Ar represents moiety (v), E and E′ represent oxygen atoms, m represents 1, w represents 1, A represents 1, B represents 1, r and s represent 0 and G represents a direct link (i.e. polyether-diphenyl-ether-phenyl-ketone-phenyl-).

Said bio-compatible polymeric material may consist essentially of one of units (a) to (f) defined above. Alternatively, said polymeric material may comprise a copolymer comprising at least two units selected from (a) to (f) defined above. Preferred copolymers include units (a). For example, a copolymer may comprise units (a) and (f); or may comprise units (a) and (e).

Said bio-compatible polymeric material preferably comprises, more preferably consists essentially of, a repeat unit of formula (XX)

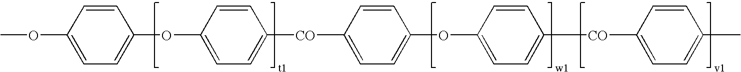

where t1, and w1 independently represent 0 or 1 and v1 represents 0, 1 or 2. Preferred polymeric materials have a said repeat unit wherein t1=1, v1=0 and w1=0; t1=0, v1=0 and w1=0; t1=0, w1=, v1=2; or t1=0, v1=1 and w1=0. More preferred have t1=1, v1=0 and w1=0; or t1=0, v1=0 and w1=0. The most preferred has t1=1, v1=0 and w1=0.

In preferred embodiments, said bio-compatible polymeric material is selected from polyetheretherketone, polyetherketone, polyetherketoneetherketoneketone and polyetherketoneketone. In a more preferred embodiment, said polymeric material is selected from polyetherketone and polyetheretherketone. In an especially preferred embodiment, said polymeric material is polyetheretherketone.

Said radiopaque material may be any material which when added to the bio-compatible polymeric material increases the radiopacity of the combination. Said radiopaque material preferably improves the imageability of the bio-compatible polymeric material when imaged using both CT and MRI techniques.

Said radiopaque material may comprise a metal, an inorganic material or an iodine-containing organic material.

Said radiopaque material may comprise a metal selected from barium, bismuth, tungsten, gold, titanium, iridium, platinum, rhenium or tantalum; a compound, for example a salt incorporating one of the aforesaid metals; a radiodense salt; or an iodine-containing organic material.

In one embodiment, said radiopaque material may include oxygen moieties. Said radiopaque material may be a ceramic. It may be a zirconium ceramic salt. Said radiopaque material preferably includes zirconium moieties and oxygen moieties. Said material may be an oxide which comprises zirconium moieties. Said material may be zirconium dioxide.

Said radiopaque material preferably has a decomposition temperature which is greater than 300° C., suitably greater than 325° C., preferably greater than 350° C., more preferably greater than 500° C., especially greater than 700° C., suitably so it can be melt-processed with the preferred bio-compatible polymeric materials.

Said radiopaque material preferably comprises a metal selected from those described or a compound for example a salt incorporating one of said metals, provided said compound has a decomposition temperature of greater than 350° C., preferably of greater than 500° C.

Said fiducial marker may include one or a plurality of bio-compatible polymeric materials. Where said marker includes a second or subsequent bio-compatible polymeric material, the second or subsequent material may have any feature of said bio-compatible polymeric material described herein.

The sum of the wt % of all organic polymeric materials (including said bio-compatible polymeric material and any additional bio-compatible polymeric materials) in said fiducial marker is preferably in the range 50 to 80 wt %, more preferably 55-75 wt %.

Said fiducial marker may include one or a plurality of radiopaque materials. In this case, each radiopaque material may independently be as described herein.

The sum of the wt % of all radiopaque materials in said fiducial marker may be in the range 20 to 80 wt %, suitably 20 to 70 wt %, preferably 20 to 55 wt %, more preferably in the range 20 to 50 wt %, especially 25 to 50 wt %.

The sum of the wt % of all organic polymeric materials and all radiopaque materials in same fiducial marker is suitably at least 80 wt %, preferably at least 90 wt %, more preferably at least 95 wt %, especially at least 99 wt %.

In a first embodiment said fiducial marker may comprise a radiopaque material in particulate form dispersed within, preferably throughout, said bio-compatible polymeric material. Said fiducial marker preferably has a substantially constant density throughout. Said marker is preferably substantially homogenous. Suitably, said polymeric material defines a matrix in which particles of radiopaque material are substantially uniformly dispersed and embedded.

The total wt % of all particulate radiopaque materials in said marker may be at least 14 wt %, suitably at least 20 wt %, preferably at least 25 wt %, more preferably at least 30 wt %, especially at least 35 wt %. The total may be 70 wt % or less, suitably less than 60 wt %, preferably less than 55 wt %. If too much radiopaque material is included the integrity and/or strength of the marker may be compromised; if there is too little, the marker may not be satisfactorily visible in for example CT or MRI imaging techniques.

The total wt % of all bio-compatible polymeric materials in said marker may be at least 40 wt %, preferably at least 50 wt %. The total may be less than 85 wt %, preferably less than 70 wt %, more preferably less than 65 wt %.

The sum of the wt % of all particulate radiopaque materials and all bio-compatible polymeric materials in said marker may be at least 80 wt %, preferably at least 90 wt %, more preferably at least 95 wt %, especially at least 99 wt %.

In a preferred example of said first embodiment, said fiducial marker includes 40 to 75 wt % of bio-compatible polymeric material (preferably of formula [XX] above, especially polyetheretherketone) and 25 to 60 wt % of radiopaque material (especially particulate material, for example a metal salt such as a barium salt). In an especially preferred example, a fiducial marker includes 45 to 70 wt % of polyetheretherketone and 30 to 55 wt % of a particulate radiopaque material, especially barium sulphate.

In another preferred example of said first embodiment, said fiducial marker includes 60 to 85 wt % of bio-compatible polymeric material (preferably of formula [xx] above, especially polyetheretherketone) and 15 to 40 wt % of a radiopaque material (especially particulate material, for example a bismuth compound for example a bismuth salt such as bismuth trioxide or bismuth oxychloride). In preferred examples, said fiducial marker includes 15-30 wt % of a bismuth compound as aforesaid and 70-85 wt % of a polyaryletherketone, especially polyetheretherketone.

As an alternative, in the aforementioned preferred examples, the salt may be replaced by a zirconium salt, for example zirconium dioxide.

In a second embodiment, a wire, for example a metal wire may be encapsulated in said bio-compatible polymeric material. The wire may have a diameter in the range 10 to 200 μm, suitably 20 to 100 μm, more preferably 25 to 75 μm, especially about 50 μm. The wire may be metal, for example selected from tantalum or another radiopaque wire. In a preferred embodiment, the wire is selected from stainless steel, tungsten and tantalum. Because the wire is very fine and is encapsulated in an inert and strong bio-compatible polymeric material, the level of underdesirable artefacts noticeable on imaging may be significantly less than when thicker wire is used; and the bio-compatible polymeric material maintains the integrity of the marker.

In a preferred example of said second embodiment, a metal wire having a diameter in the range 0.1 mm to 0.4 mm (preferably in the range 0.1 mm to 0.3 mm) and preferably being selected from stainless steel, tungsten and tantalum defines a core which is encapsulated in a bio-compatible polymeric material as described herein (preferably one of formula [xx] and especially polyetheretherketone), wherein the bio-compatible polymeric material is filled with a radiopaque material, especially a metal salt, with barium and bismuth salts (e.g. barium sulphate, bismuth trioxide and bismuth oxychloride) being especially preferred. The layer which encapsulates the wire may include 40 to 85 wt % of said bio-compatible polymeric material and 15 to 60 wt % of filler (e.g. one or more radiopaque fillers as described). When a barium salt is included, the layer may include 40 to 70 wt % (preferably 45 to 60 wt %) of said salt with the balance being said bio-compatible polymer. When a bismuth salt is included, the layer may include 15 to 40 wt % (preferably 15 to 30 wt %, more preferably 18 to 28 wt %) of said bismuth salt.

As an alternative, in a preferred example of said second embodiment, said radiopaque material may comprise zirconium oxide.

In a third embodiment, said fiducial marker may comprise bio-compatible polymeric material and fibrous radiopaque material. Such a marker may be made using a pultrusion technique.

In a fourth embodiment, a fiducial marker may comprise first and second fillers encapsulated in said bio-compatible polymeric material, which may be of formula [xx] and is preferably polyetheretherketone. A first filler may be a metal, suitably in powderous form, which may be selected from stainless steel, tantalum and titanium. A second filler may be a radio dense salt, suitably as described herein, with barium salts and bismuth salts being preferred examples. Said fiducial marker may include 5-20 wt % of said first filler 15-60 wt % of said second filler and 20-80 wt % of said bio-compatible polymeric material. When said marker includes a bismuth salt, it may include 5-20 wt % of said first filler 15 to 40 wt % (preferably 15 to 30 wt %, more preferably 18 to 28 wt %) of said bismuth salt and the balance being said bio-compatible polymeric material. When said marker includes a barium salt, it may include 5-20 wt % of said first filler, 40-70 wt % (preferably 45-60 wt %) of said salt, with the balance being said bio-compatible polymeric material.

According to a second aspect of the invention, there is provided the use of a member which comprises a radiopaque material encapsulated in a bio-compatible polymeric material as a fiducial marker.

The member may be a fiducial marker as described in said first aspect.

According to a third aspect of the invention, there is provided the use of a radiopaque material encapsulated in a bio-compatible polymeric material in the manufacture of a fiducial marker for use in marking a position on a human or animal body.

The fiducial marker may be as described according to said first aspect.

According to a fourth aspect of the invention, there is provided a method of marking a position in the human or animal body, the method comprising positioning, preferably securing, within the body a fiducial marker as described according to the first aspect.

The method may include positioning a plurality, preferably at least four, markers in the body.

According to a fifth aspect of the invention, there is provided a method of obtaining images of predetermined positions of a human or animal body, the method comprising imaging a human or animal body in which has been positioned one or a plurality (preferably a plurality) of fiducial markers according to said first aspect.

The method may include imaging the body by CT or MRI scanning techniques. Preferably, the method involves imaging by both CT and MRI scanning techniques. The method may involve X-ray imaging. Advantageously, the fiducial markers are visible to X-ray imaging and compatible with CT and MRI methods.

The method may include the step of positioning one or a plurality of said fiducial markers in position within the body prior to said imaging.

According to a sixth aspect of the invention, there is provided a method of making a fiducial marker, the method comprising encapsulating a radiopaque material in a biocompatible material.

The method preferably includes the step of extrusion to encapsulate said radiopaque material. A mixture comprising radiopaque and polymeric materials may be extruded suitably to define a filament. Alternatively, a wire may be coated with extruded polymeric material.

The method may include chopping extruded material to define fiducial markers of appropriate dimensions.

The invention extends to a pack comprising a fiducial marker according to said first aspect contained in a packaging material. The packaging material could be sterile.

Preferably fiducial markers described herein are for use and/or use in relation to human bodies.

Any feature of any aspect of any invention or embodiment described herein may be combined with any feature of any aspect of any other invention or embodiment described herein mutatis mutandis.

### BRIEF DESCRIPTION OF THE DRAWINGS

Specific embodiments of the invention will now be described by way of example, with reference to the accompanying drawings, in which

FIGS. 1 (a) to (c) are CT images of different fiducial markers; and

FIGS. 2(a) and (b) are MRI images of different fiducial markers.

### DETAILED DESCRIPTION

The following is referred to hereinafter:

PEEK OPTIMA LT3 polymer refers to polyetheretherketone obtained from Invibio Limited, UK.

In Example 1 hereinafter the preparation of fiducial markers comprising polyetheretherketone and barium sulphate is described. Such markers are compared to known metal markers in CT-imaging, MRI-imaging and X-ray imaging in the following examples.

### EXAMPLE 1

### Preparation of polyetheretherketone-Based Fiducial Markers

PEEK OPTIMA LT3 polymer and a highly pure grade of barium sulphate comprising greater than 98% of particles 10 μm or less were compounded in a twin screw melt extrusion compounder and a lace produced of 2-3 mm diameter. The lace was passed to a conveyor, cooled and then chopped into granules. The granules were then introduced into an extruder and monofilaments produced which were then chopped to produce fiducial markers of predetermined lengths comprising polyetheretherketone polymer with barium sulphate dispersed substantially homogenously throughout the polymer.

### EXAMPLES 2 TO 16 AND C1 TO C4

Following the procedure described in Example 1, fiducial markers having different levels of barium sulphate and/or different dimensions were prepared as shown in Table 1.

TABLE 1

| | | Amount | Diameter | Length |
|---|---|---|---|---|
| | Amount | barium | of | of |
| Example | polyetheretheketone | sulphate | marker | marker |
| No | (wt %) | (wt %) | (mm) | (mm) |
| 2 | 94 | 6 | 1.5 | 2 |
| 3 | 94 | 6 | 1.5 | 3 |
| 4 | 94 | 6 | 1.5 | 4 |
| 5 | 90 | 10 | 1.5 | 2 |
| 6 | 90 | 10 | 1.5 | 3 |
| 7 | 90 | 10 | 1.5 | 4 |
| 8 | 80 | 20 | 1.5 | 2 |
| 9 | 80 | 20 | 1.5 | 3 |
| 10 | 80 | 20 | 1.5 | 4 |
| 11 | 70 | 30 | 1.5 | 2 |
| 12 | 70 | 30 | 1.5 | 3 |
| 13 | 70 | 30 | 1.5 | 4 |
| 14 | 80 | 20 | 0.9 | 2 |
| 15 | 80 | 20 | 0.9 | 3 |
| 16 | 80 | 20 | 0.9 | 4 |

The markers of examples 2 to 16 were compared to conventional metal wire markers as described in Table 2.

TABLE 2

| | | Diameter of | Length of |
|---|---|---|---|
| Example No | Metal | marker (mm) | marker (mm) |
| C1 | Pt | 0.9 | 2 |
| C2 | Pt | 0.9 | 3 |
| C3 | Pt | 0.9 | 4 |
| C4 | Au | 1.0 | 5 |

The markers of Examples 2 to 16, and C1 to C4 were assessed by CT-imaging. In each case it was found that the markers of Examples 2 to 16 produced very significantly fewer artefacts compared to the metal markers.

### EXAMPLES 17, 18, C5 AND C6

### Comparison of polyetheretherketone-Based Markers and Metal Markers in Various Imaging Systems

Fiduciary markers described in Table 3 were assessed in various imaging systems.

TABLE 3

| | | Amount | | Diameter | Length |
|---|---|---|---|---|---|
| | Amount | barium | | of | of |
| Example | polyetheretherketone | sulphate | | marker | marker |
| No | (wt %) | (wt %) | Metal | (mm) | (mm) |
| 17 | 70 | 30 | — | 0.9 | 4 |
| 18 | 70 | 30 | — | 1.5 | 4 |
| C5 | — | — | Au | 0.9 | 4 |
| C6 | — | — | Pt | 0.9 | 4 |

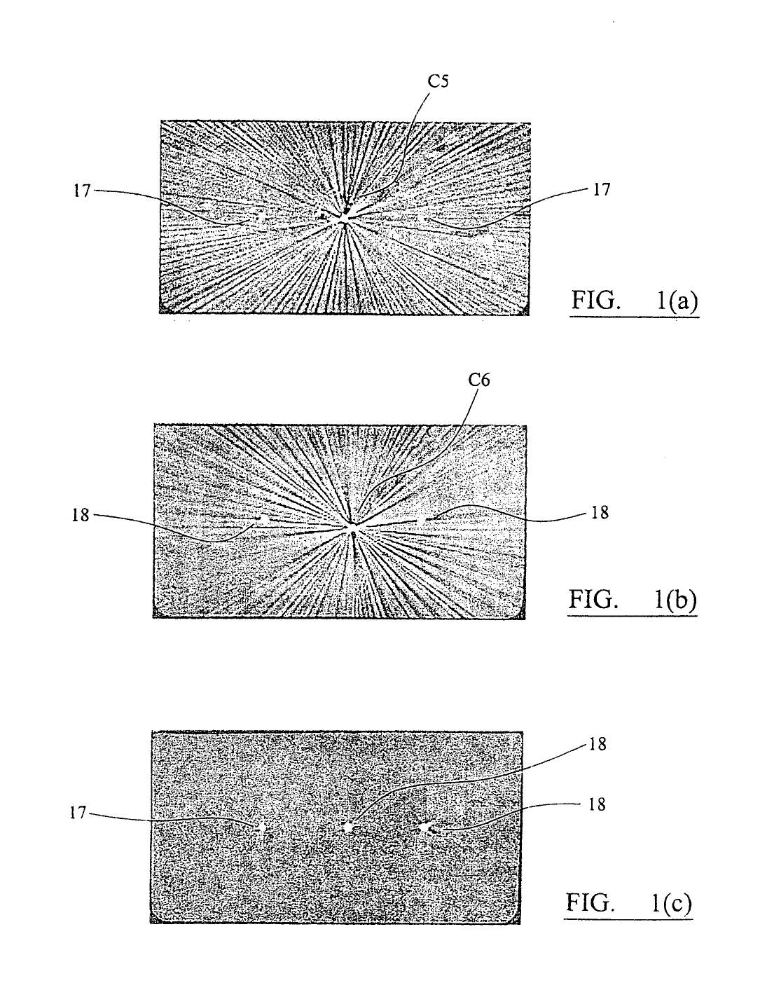

Referring to FIG. 1(a), the central spot is the CT image of Example C5 from which it will be noted that there is a significant level of distortion and a significant starburst effect, in comparison to the two Example 17 markers which are nonetheless still clearly visible.

Similarly, referring to FIG. 1(b), the Example C6 marker is substantially distorted and has produced a significant starburst effect compared to the two Example 18 markers.

FIG. 1(c) illustrates changes in the images when wider diameter markers are used (compare Examples 17 and 18 and note that each of the markers is highly visible and has significantly less distortion compared to the marker of Examples C5 and C6 of FIGS. 1(a) and 1(b).

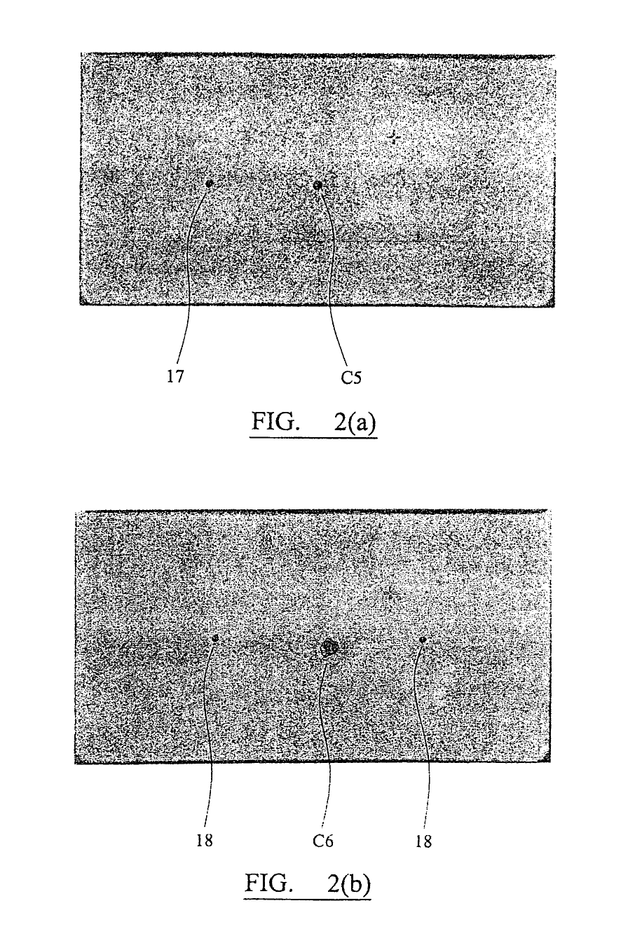

Referring to FIG. 2(a) it will be noted that in MRI imaging the polyetheretherketone-based marker of example 17 includes little distortion and has intensity which is comparable to that of the gold marker of Example C5. Referring to FIG. 2(b), the distortion of the platinum marker of Example C6 will be noted compared to that of the two Example 18 markers.

In some cases, for example where CT and/or MRI equipment is not available, conventional X-ray imaging may be used to view markers. Whilst the markers of Examples 17 and 18 are less visible under X-ray imaging than both platinum and gold markers, they can still readily be detected, especially when their image is enhanced by conventional image processing techniques.

Thus, markers described herein can be imaged using CT, MRI and X-ray techniques. In each case, images include less distortion and/or starburst and/or other artefacts compared to metal, for example gold of platinum, markers.

Markers as described may be provided in a range of dimensions as shown in the table below. Furthermore, spherical markers, having diameters in the range 1 to 5 mm may be provided.

| Diameter of marker (mm) | Length (mm) |
|---|---|
| 0.8 | 3 |
| 0.8 | 5 |
| 0.8 | 7 |
| 1.0 | 3 |
| 1.2 | 3 |
| 1.0 | 5 |
| 1.0 | 7 |

### EXAMPLE 19

Using a standard wire coating technique a 0.12 mm diameter stainless steel wire was coated with a homogenous mixture comprising PEEK OPTIMA LT3 polymer (50 wt %) and the barium sulphate referred to in previous examples (50 wt %). The coated wire was then cut to size to define a fiducial marker comprising a wire core and an outer homogenous sheath of PEEK OPTIMA LT3 polymer and barium sulphate.

The inclusion of the wire core improves visibility of the marker under MRI conditions, whilst the barium sulphate improves the visibility of the marker in other imagining techniques.

As variations on the example, the stainless steel wire core may be replaced with tantalum or titanium; the amount of barium sulphate may be adjusted (e.g. in the range 30-70 wt %) or; alternate radio dense materials may be used instead of barium sulphate. For example, a bismuth salt (e.g. bismuth trioxide or bismuth oxychloride) may be used at a level of 15-45 wt % with 55-85 wt % of the polymer.

### EXAMPLE 20

As an alternative to the Example 19 embodiment, the metal wire may be replaced with metal powder, for example of stainless steel, tungsten or tantalum, at up to 20 wt % of the entire marker. An example of such a marker may include up to 20 wt % of metal powder, 45 to 70 wt % of barium sulphate (or 15-45 wt % of a bismuth salt if such a salt is used instead of the barium sulphate) and the balance being PEEK OPTIMA LT3. The materials are mixed to define a homogenous mass and extruded to define an elongate marker having a diameter of 1 mm.

In a further embodiment, a fiducial marker may be prepared by selection of a polyetheretherketone (PEEK OPTIMA LT3 polymer obtained from Invibio Limited, UK) and a highly pure grade of zirconium dioxide of suitable particle size. The combination may then be introduced into a twin screw melt extrusion compounder and a lace produced having a diameter of 2 to 3 mm. The lace may then be passed to a conveyer, cooled and chopped into granules. The granules may be introduced into an extruder and monofilaments produced which may then be chopped to produce fiducial markers of predetermined lengths comprising polyetheretherketone polymer with zirconium dioxide substantially homogenously dispersed throughout the polymer.

The zirconium dioxide-based markers described can be imaged using CT, MRI and X-ray techniques. In each case, images include less distortion and/or starburst and/or other artefacts compared to other known markers.

Zirconium dioxide markers as described may be provided in a range of dimensions as shown in the table below. Furthermore, spherical markers, having diameters in the range 1 to 5 mm may be provided.

| Diameter of marker (mm) | Length (mm) |
|---|---|
| 0.8 | 3 |
| 0.8 | 5 |
| 0.8 | 7 |
| 1.0 | 3 |
| 1.2 | 3 |
| 1.0 | 5 |
| 1.0 | 7 |

In another embodiment, a standard wire coating technique may be used to coat a 0.12 mm diameter stainless steel wire with a homogenous mixture comprising PEEK OPTIMA LT3 polymer (50 wt %) and zirconium dioxide (50 wt %). The coated wire may be cut to size to define a fiducial marker comprising a wire core and an outer homogenous sheaf of PEEK OPTIMA LT3 polymer and zirconium dioxide.

The inclusion of the wire core improves visibility of the marker under MRI conditions, whilst the zirconium dioxide improves the visibility of the marker in other imagining techniques.

As variations on the example, the stainless steel wire core may be replaced with tantalum or titanium; or the amount of zirconium dioxide may be adjusted (e.g. in the range 30-70 wt %).

In a further alternative, the metal wire may be replaced with metal powder, for example of stainless steel, tungsten or tantalum, at up to 20 wt % of the entire marker. An example of such a marker may include up to 20 wt % of metal powder, 30 to 70 wt % of zirconium dioxide and the balance being PEEK OPTIMA LT3. The materials are mixed to define a homogenous mass and extruded to define an elongate marker having a diameter of 1 mm.

1. A fiducial marker which comprises a radiopaque material encapsulated in a bio-compatible polymeric material.

2. A marker according to claim 1, wherein said marker has a maximum dimension measured in a first direction of less than 50 mm.

3. A marker according to claim 1, which has a maximum dimension measured in a first direction of less than 10 mm.

4. A marker according to claim 3, wherein said marker has a dimension in a second direction perpendicular to the first direction which is less than said maximum dimension in said first direction.

5. A marker according to claim 1 which has a volume of less than 20 mm³.

6. A marker according to claim 5, which has a density of less than 3.5 g/cm³ and greater than 1.2 g/cm³.

7. A marker according to claim 1, wherein said marker is elongate or spherical.

8. A marker according to claim 1, which includes substantially no void areas.

9. A marker according to claim 1, wherein said radiopaque material is substantially immovably fixed in position in said marker so that its position relative to that of the polymeric material is substantially immovably fixed.

10. A marker according to claim 1, wherein said radiopaque material is substantially fully enclosed by said bio-compatible polymeric material.

11. A marker according to claim 1, which comprises radiopaque material and polymeric material which have been extruded.

12. A marker according to claim 1, which has a weight of at least 3 mg and less than 100 mg.

13. A marker according to claim 1, which includes at least 3 wt % and less than 80 wt % of radiopaque material.

14. A marker according to claim 1, which includes at least 30 wt % of radiopaque material.

15. A marker according to claim 1, which includes at least 30 wt % of bio-compatible polymeric material.

16. A marker according to claim 1, which includes at least 50 wt % of bio-compatible polymeric material.

17. A marker according to claim 1, wherein the sum of the wt % of said bio-compatible polymeric material and said radiopaque material in said fiducial marker is at least 80 wt %.

18. A marker according to claim 1, said bio-compatible material having a Notched Izod Impact Strength (Specimen 80 mm×10 mm×4 mm with a cut 0.25 mm notch (Type A), tested at 23° C., in accordance with ISO180) of at least 4 KJm⁻².

19. A marker according to claim 1, wherein said bio-compatible polymeric material is semi-crystalline.

20. A marker according to claim 1, wherein said bio-compatible polymeric material includes a polymeric moiety which is an acrylate, a urethane, a vinyl chloride, a silicone, a siloxane, a sulphone, a carbonate, a fluoroalkylene, an acid, an oxyalkylene, an ester or an ether.

21. A marker according to claim 1, wherein said bio-compatible polymeric material is selected from a polyalkylacrylate, a polyfluoroalkylene, a polyurethane, a polyalkylene, a polyoxyalkylene, a polyester, a polysulphone, a polycarbonate, a polyacid, a polyalkylene oxide ester, a polyvinylchloride, a silicone, a polysiloxane, a nylon, a polyaryletherketone, a polarylethersulphone, a polyether imide and any copolymer which includes any of the aforementioned.

22. A marker according to claim 1, where said bio-compatible polymeric material comprises, a repeat unit of formula (xx) 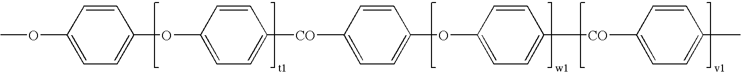
where t1, and w1 independently represent 0 or 1 and v1 represents 0, 1 or 2.

23. A marker according to claim 1, wherein said bio-compatible polymeric material is polyetheretherketone.

24. A marker according to claim 1, wherein said radiopaque material comprises a metal selected from barium, bismuth, tungsten, gold, titanium, iridium, platinum, rhenium or tantalum; a compound incorporating one of the aforesaid metals; a radiodense salt; or an iodine-containing organic material.

25. A marker according to claim 1, wherein said radiopaque material has a decomposition temperature which is greater than 300° C.

26. A marker according to claim 1, wherein said marker includes 40-75 wt % of bio-compatible polymeric material and 25-60 wt % of radiopaque material.

27. A marker according to claim 1, wherein said marker includes 1 to 20 wt % of metal, 15 to 60 wt % of one or more radiodense salts and 20-84 wt % of bio-compatible polymeric material(s).

28. A marker according to claim 27, wherein said metal defines a core which is encapsulated by said bio-compatible polymeric material or is in particulate form.

29. A marker according to claim 28, wherein said marker includes at least 5 wt % of metal and at least 35 wt % of bio-compatible polymeric material(s).

30. The use of a member which comprises a radiopaque material encapsulated in a bio-compatible polymeric material as a fiducial marker.

31. The use of a radiopaque material encapsulated in a bio-compatible polymeric material in the manufacture of a fiducial marker for use in marking a position on a human or animal body.

32. A method of marking a position in the human or animal body, the method comprising positioning within the body a fiducial marker as described in claim 1.

33. A method of obtaining images of predetermined positions of a human or animal body, the method comprising imaging a human or animal body in which has been positioned one or a plurality of fiducial markers according to claim 1.

34. A method of making a fiducial marker, the method comprising encapsulating a radiopaque material in a bio-compatible material.

35. A fiducial marker which comprises a radiopaque material encapsulated in a bio-compatible polymeric material, wherein said radiopaque material comprises a metal selected from barium, bismuth, tungsten, gold, titanium, iridium, platinum, rhenium or tantalum; a compound incorporating one of the aforesaid metals; a radiodense salt; or an iodine-containing organic material; and said bio-compatible polymeric material comprises polyetheretherketone; and wherein said marker includes 40-75 wt % of bio-compatible polymeric material and 25-60 wt % of radiopaque material.
